(19) United States
(12) Patent Application Publication
(10) Pub. No.: US 2022/0265245 A1
(43) Pub. Date: Aug. 25, 2022

(54) Computing Device Controller System

(71) Applicant: Bard Access Systems, Inc., Salt Lake City, UT (US)

(72) Inventors: Steffan Sowards, Salt Lake City, UT (US); Anthony K. Misener, Bountiful, UT (US); William Robert McLaughlin, Bountiful, UT (US)

(21) Appl. No.: 17/677,728

(22) Filed: Feb. 22, 2022

Related U.S. Application Data
(60) Provisional application No. 63/152,729, filed on Feb. 23, 2021.

(51) Int. Cl. A61B 8/00; G06F 3/0338; G06F 3/0362; G06F 3/02; A61B 46/10

(52) U.S. Cl. CPC A61B 8/469; G06F 3/0338; G06F 3/0362; G06F 3/02; A61B 46/10; A61B 8/54; A61B 8/56; G06F 2203/04108; G06F 3/044

(57) ABSTRACT

Disclosed herein is a computing device controller system including a computing device outside of a sterile field, and a controller in communication with the computing device, the controller having a controller body including an input mechanism, the input mechanism including one or both of a non-tactile input and a tactile input, wherein the input mechanism is configured to be accessible in the sterile field and to provide one or more input parameter changes to the computing device.

### PRIORITY

This application claims the benefit of priority to U.S. Provisional Application No. 63/152,729, filed Feb. 23, 2021, which is incorporated by reference in its entirety into this application.

### BACKGROUND

When a sterile field is present during a medical procedure, it can be difficult for a clinician to interact with or provide input to a computing device used in the medical procedure. The clinician must exit the sterile field, relay the clinician's input to a person external the sterile field, or configure the computing device to be within the sterile field. Some computing devices may include tactile controllers, requiring a user to exit the sterile field to use. This process can require time during the procedure and reagents for sterilization of the user each time the user exits the sterile field. It would be beneficial to the user to be able to maintain sterility within the sterile field while allowing the user to interface with or provide input to the computing device. Disclosed herein is a system and a method that address the foregoing.

### SUMMARY

Disclosed herein in some embodiments is a computing device controller system including a computing device outside of a sterile field, and a controller in communication with the computing device, the controller having a controller body including an input mechanism, the input mechanism including one or both of a non-tactile input and a tactile input, wherein the input mechanism is configured to be accessible in the sterile field and to provide one or more input parameter changes to the computing device.

In some embodiments, the input mechanism includes the non-tactile input, the non-tactile input comprising one or more capacitive induction sensors, one or more optical sensors, or both one or more capacitive induction sensors and one or more optical sensors. In some embodiments, the input mechanism includes the tactile input, the tactile input comprising a joystick or a directional pad. The controller can include one or more controls configured to provide one or more input parameter changes to the computing device. The one or more controls can be palpable controls. The one or more palpable controls can include one or more of a knob, a trigger, and a button. The one or more controls can be visually identifiable.

In some embodiments, the controller body includes an attachment connection port, having one or more attachment connectors configured to couple to one or more attachments within the sterile field. The one or more attachments can include an ECG module, a stylet, a magnet tracking sensor, an electromagnetic tracking sensor, an impedance driver, an impedance receiver, a fiber optic interrogator, an RFID reader, and combinations thereof. In some embodiments, the controller is configured to transmit data from the attachment to the computing device.

In some embodiments, the sterile field is defined by a sterile drape. The controller can be below the sterile field and/or shrouded within a sterile sheath. In some embodiments, the controller is in wireless communication with the computing device. In some embodiments, the one or more controls are visually identifiable through a clear barrier or by the one or more controls being illuminated. In some embodiments, the controller is fiber optic enabled.

In some embodiments, the controller includes a console having one or more processors, non-transitory computer readable medium and a plurality of logic modules. The plurality of logic modules when activated by the one or more processors may be configured to perform one or more of: receiving input from the non-tactile input mechanism; correlating input from the non-tactile input mechanism with input parameter changes on the computing device; receiving input from the one or more controls; correlating input from the controls with input parameter changes on the computing device; transmitting the input parameter changes to the computing device; and illuminating the non-tactile input mechanism and controls.

In some embodiments, the computing device includes an ultrasound system. In some embodiments the controller includes an attachment connection port having one or more attachment connectors configured to receive one or more attachment inputs from the attachment within the sterile field.

Disclosed herein is also a method of providing input parameter changes to a computing device while maintaining sterility in a sterile field, including placing a controller in communication with a computing device outside of a sterile field; placing the controller near the sterile field; and inputting input parameter changes to the computing device from the sterile field. In some embodiments, placing the controller in communication with the computing device outside of the sterile field includes placing the controller in wireless communication with the computing device. In some embodiments, placing the controller in communication with the computing device outside of the sterile field includes coupling the controller to the computing device. In some embodiments, placing the controller near the sterile field includes placing the controller within a sterile sheath and/or placing the controller below the sterile field.

In some embodiments, providing input parameter changes to the computing device from the sterile field includes providing input parameter changes to the computing device through an input mechanism of the controller. The input mechanism can be a tactile input or a non-tactile input. The tactile input can include a joystick or a directional pad. The non-tactile input can include one or more capacitive induction sensors, one or more optical sensors, or both one or more capacitive induction sensors and one or more optical sensors.

In some embodiments, providing input parameter changes to the computing device from the sterile field includes providing input parameter changes to the computing device through one or more controls. The one or more controls can be palpable controls. The one or more palpable controls can include one or more of a knob, a trigger, and a button.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a perspective view of a computing device controller system, in accordance with some embodiments

FIGS. 2A-2B illustrate perspective views of embodiments of a controller, in accordance with some embodiments.

FIG. 2C illustrates a side view of the controller, in accordance with some embodiments.

FIG. 3 illustrates a block diagram of some components of the controller including the console, in accordance with some embodiments.

FIGS. 4A-4C illustrate perspective views of embodiments of the controller, in accordance with some embodiments.

FIGS. 5A-5B illustrate an exemplary method of using the controller while maintaining sterility in a sterile field, in accordance with some embodiments.

FIG. 6 illustrates a perspective view of the controller within a sterile sheath, in accordance with some embodiments.

FIG. 7 illustrates a block diagram of an exemplary method of providing input parameter changes to a computing device while maintaining sterility in a sterile field, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The term “computing device” should be construed as electronics with the data processing capability and/or a capability of connecting to any type of network, such as a public network (e.g., Internet), a private network (e.g., a wireless data telecommunication network, a local area network “LAN”, etc.), or a combination of networks. Examples of a computing device may include, but are not limited or restricted to, the following: a server, an endpoint device (e.g., a laptop, a smartphone, a tablet, a “wearable” device such as a smart watch, augmented or virtual reality viewer, or the like, a desktop computer, a netbook, a medical device, or any general-purpose or special-purpose, user-controlled electronic device), a mainframe, internet server, a router; or the like.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical, or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

Unless defined otherwise, all technical and scientific terms used herein have the same meaning as commonly understood by those of ordinary skill in the art.

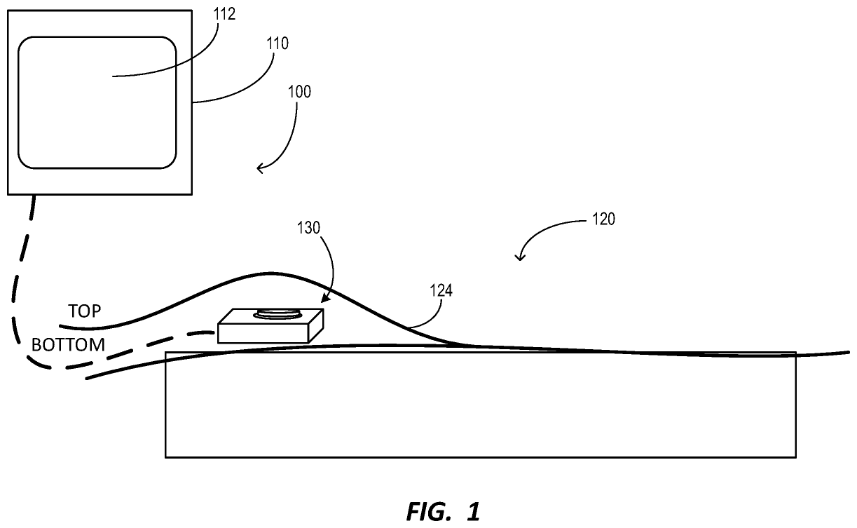

FIG. 1 illustrates a perspective view of a computing device controller system (“system”) 100, in accordance with some embodiments. In some embodiments, the system 100 includes a computing device 110 outside of a sterile field 120, in communication with a controller 130. In some embodiments, the computing device 110 may be in communication with a display 112. In some embodiments, the display 112 may be physically separate from the computing device 110 or may be physically combined with the computing device 110, as illustrated in FIG. 1. In some embodiments, the computing device 110 may include an ultrasound system. In some embodiments, the controller 130 may be near the sterile field 120 or within the sterile field 120. In some embodiments, the controller 130 may be configured to transmit various input parameters to the computing device 110 that may be depicted on the display 112. In some embodiments, the controller 130 may be wired to the computing device 110 or may be in wireless communication with the computing device 110. Exemplary wireless communication modalities can include WiFi, Bluetooth, Near Field Communications (NFC), cellular Global System for Mobile Communication (“GSM”), electromagnetic (EM), radio frequency (RF), combinations thereof, or the like.

In some embodiments, the controller 130 may be configured to be near the sterile field 120. In some embodiments, near the sterile field includes below the sterile field. In some embodiments, a top of a sterile drape 124 may be configured to define the sterile field 120. Outside of the sterile field 120 may include below the sterile field 120, including below the sterile drape 124. In some embodiments, the controller 130 may be configured to be within the sterile field 120 by being sheathed within a sterile sheath, as will be described in more detail herein. In some embodiments, the controller 130 may be covered by the sterile drape 124 but still be accessible to a user, by touching the controller 130 or through other means, through the sterile drape 124 without disrupting or leaving the sterile field 120, as will be described in more detail herein.

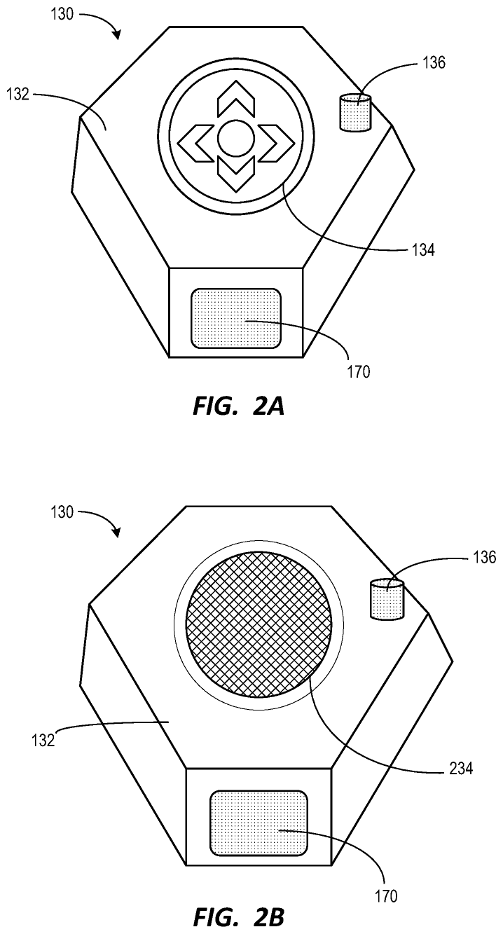

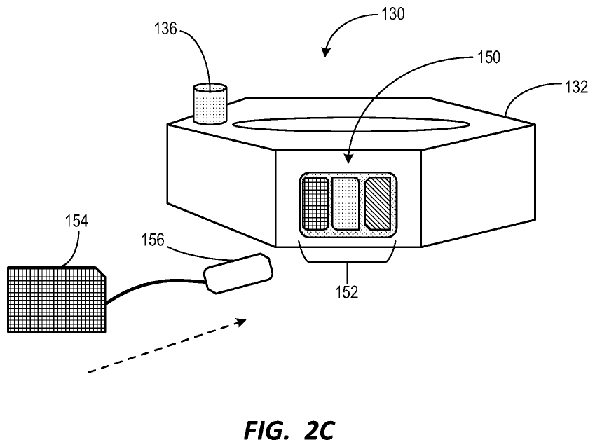

FIGS. 2A-2B illustrate perspective views of the controller 130, in accordance with some embodiments. As illustrated in FIG. 2A, in some embodiments, the controller 130 includes a controller body 132, having a top side and a bottom side. In some embodiments, the top side may be covered by the sterile drape 124. In some embodiments, the controller body 132 may include a rectangular prism, a triangle prism, a pentagonal prism, a hexagonal prism, a cube or the like. In some embodiments, the bottom side may be configured to detachably couple through an adhesive compound, hook and loop fastener or the like to the sterile drape 124, a table, a tray, a stand or the like. In some embodiments, the top side of the controller body 132 includes an input mechanism 134. In some embodiments, the input mechanism 134 may be a tactile input mechanism 134, configured to allow the user, through touch, to provide input parameter changes to the computing device 110 or may be a non-tactile input mechanism 134, configured to allow the user, through other means, to provide input parameter changes to the computing device 110 that will be described in more detail herein. In some embodiments, the tactile input mechanism 134 may include a joystick, a directional pad, a trigger or the like, as will be described in more detail herein. In some embodiments, the top side of the controller body 132 may also include one or more controls 136. In some embodiments, the one or more controls 136 may be configured to be visually identifiable. In some embodiments, the one or more controls 136 may include palpable controls, extending from the top side of the controller body 132. In some embodiments, the one or more palpable controls 136 may include a knob, a button or the like. In some embodiments, the tactile input mechanism 134 may be configured to control a first set of input parameters and the one or more controls 136 may be configured to control a second set of input parameters. In some embodiments, the tactile input mechanism 134 and the one or more controls 136 may be configured to control both the first and second set of input parameters. In some embodiments, the controller body 132 may include a computing device port 170, configured to couple the controller 130 to the computing device 110.

In some embodiments, the controller may include the non-tactile input mechanism 134 including a capacitive detection sensor, an optical detection sensor or the like. In an embodiment, as illustrated in FIG. 2B, the non-tactile input mechanism 134 may include the one or more capacitive detection sensors 234 configured to detect changes in an electrical field above the one or more capacitive detection sensors 234 and associate the changes in the electrical field with input parameter changes in the computing device 110. In this embodiment, the controller 130 may be placed under a sterile drape 124 or within a sterile sheath and the user may provide input parameter changes to the computing device 110 by placing a hand or a limb over the one or more capacitive detection sensor 234 while maintaining sterility within the sterile field 120. Once the user's hand or limb is placed over the one or more capacitive detection sensors 234, the user's hand or limb may move within the electrical field to change the input parameters for the computing device 110. In some embodiments, placing the hand over the one or more capacitive detection sensors 234 includes hovering the hand over the one or more capacitive detection sensors 234. Advantageously, the one or more capacitive detection sensors 234 allows the user to provide input parameter changes to the computing device 110 without physically contacting the controller body 132, maintaining the sterility of the sterile field 120.

In some embodiments as illustrated in FIG. 2C, the controller body 132 may include an attachment connection port 150, having one or more attachment connectors 152. In some embodiments, the attachment connection port 150 may be configured to receive one or more attachment inputs 156 of an attachment 154 into the one or more attachment connectors 152. In some embodiments, the one or more attachments 154 may be within the sterile field 120. In some embodiments, the attachments 154 may include an ECG attachment, a stylet, a magnet tracking sensor, an electromagnetic tracking sensor, an impedance driver, an impedance receiver, a fiber optic interrogator, an RFID reader, an ECG module or the like. For example, the attachment connection port 150 may be configured to receive a fiber optic input from an attachment 154 having fiber optics therein, an ECG input from an ECG attachment or a stylet connection from a stylet. In some embodiments, the data received from each attachment may be configured to be transmitted from the controller 130 to the computing device 110. In some embodiments, the attachments 154 may be utilized within the sterile field 120. Advantageously, each attachment may be connected to the controller body 132, reducing the number of cables required for each attachment 154.

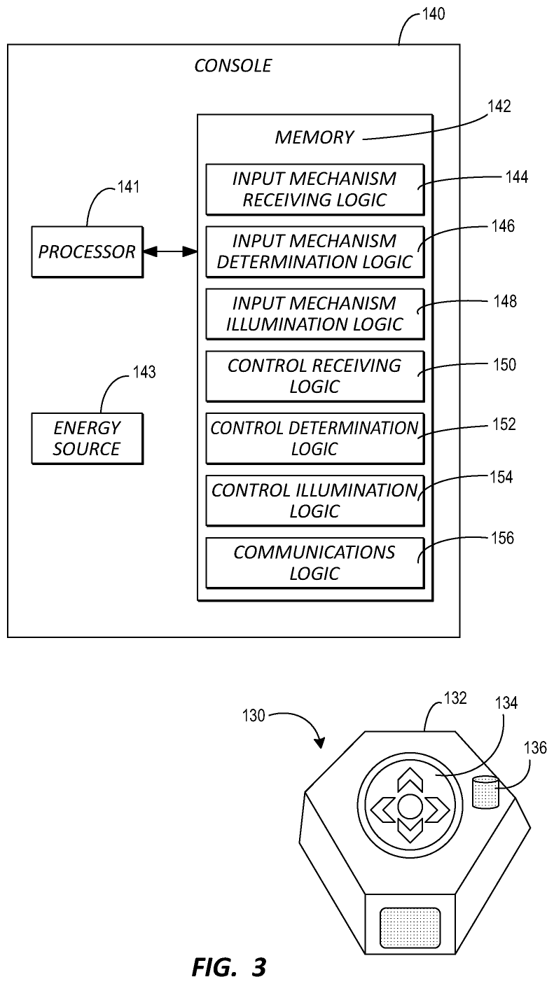

FIG. 3 illustrates a perspective view of various components of the system 100, in accordance with some embodiments. In some embodiments, the controller 130 may include a console 140 having one or more processors 141, an energy source 143, non-transitory computer readable medium (“memory”) 142 and a plurality of logic modules. In some embodiments, the console 140 may be located within the controller body 132. In some embodiments, the plurality of logic modules may include one or more of: an input mechanism receiving logic 144, an input mechanism determination logic 146, an input mechanism illumination logic 148, a palpable control receiving logic 150, a control determination logic 152, a palpable control illumination logic 154, and a communications logic 156. In some embodiments, the input mechanism receiving logic 144 receives data inputs from the input mechanism 134. In some embodiments, the data input may include the physical state of the tactile input mechanism (e.g., physical location of the joystick) or the state of the non-tactile input mechanism (e.g., state of the tactile input mechanism 134. In some embodiments, the input mechanism determination logic 146 correlates the data input from the input mechanism 134 with one or more input parameter changes on the display 112 of the computing device 110.

In some embodiments, the input mechanism illumination logic 148 may be configured to illuminate the tactile input mechanism 134 or non-tactile input mechanism 134 for ease of use by the user. In some embodiments, the control receiving logic 150 may be configured to receive the data input correlated to the physical state of the one or more controls 136, including when the one or more controls 136 are palpable controls. In some embodiments, the control determination logic 152 may be configured to correlate the data input from the physical state of the control 136 with one or more parameter changes or set of parameter changes on the computing device 110 or depicted on the display 112. In some embodiments, the control illumination logic 154 may be configured to illuminate the one or more controls 136 so that the one or more controls 136 are visually identifiable. In some embodiments, the control illumination logic 154 may be configured to illuminate the one or more controls 136, each a first color or a second color. In some embodiments, the communications logic 156 may be configured to transmit the data input from the tactile input mechanism 134 or non-tactile input mechanism 134 and the one or more controls 136 to the computing device 110.

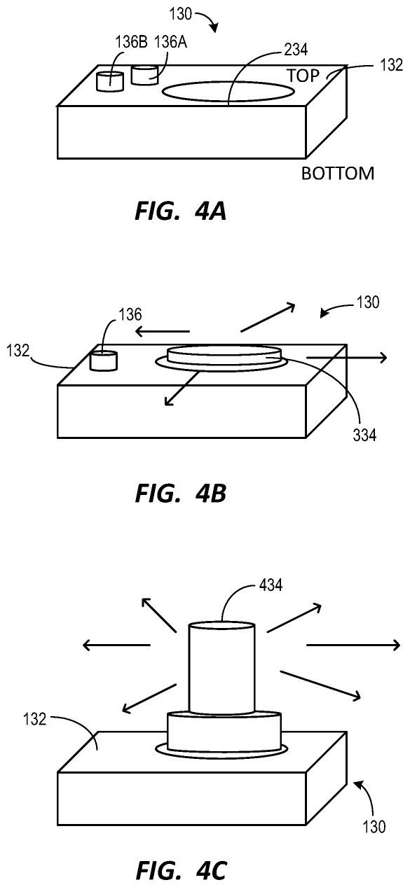

FIGS. 4A-4C illustrate perspective views of the controller 130, in accordance with some embodiments. As illustrated in FIG. 4A, in some embodiments, the controller 130 includes the capacitive detection sensor 234 and a first palpable control 136A and a second palpable control 136B. In some embodiments, the first palpable control 136A and the second palpable control 136B may be configured to be illuminated, indicating to the user the location and status of the first palpable control 136A and the second palpable control 136B, when the sterile drape 124 is covering the controller 130. As illustrated in FIG. 4B, the tactile input mechanism 134 may include a directional pad 334. In some embodiments, the directional pad 334 may be configured to be illuminated, to indicate to the user the directions on the directional pad 334. In some embodiments, the directional pad 334 may be configured to provide input parameters to the computing device 110. In some embodiments, the directional pad 334 may be configured to be touch sensitive, wherein physical contact with the directional pad 334 provides parameter inputs for the computing device 110. In some embodiments, the directional pad 334 may require physical force upon the directional pad 334 in order to provide input parameters for the computing device 110. In some embodiments, the one or more palpable controls 136 and the directional pad 334 may be configured to be illuminated through the sterile drape 124.

As illustrated in FIG. 4C, in some embodiments, the tactile input mechanism 134 may include a joystick 434. The joystick 434 may be configured to provide 360 degrees of parameter inputs that may be correlated with parameter inputs for the computing device 110. For example, in some embodiments, the parameter inputs may be correlated to (X,Y) coordinates of a cursor depicted on the display 112. In some embodiments, a part of or the entire joystick 434 may be configured to be illuminated. The joystick 434 may be configured to extend from the top side of the controller body 132, allowing the user to grasp and control the joystick 434, while the controller 130 is below the sterile field 120.

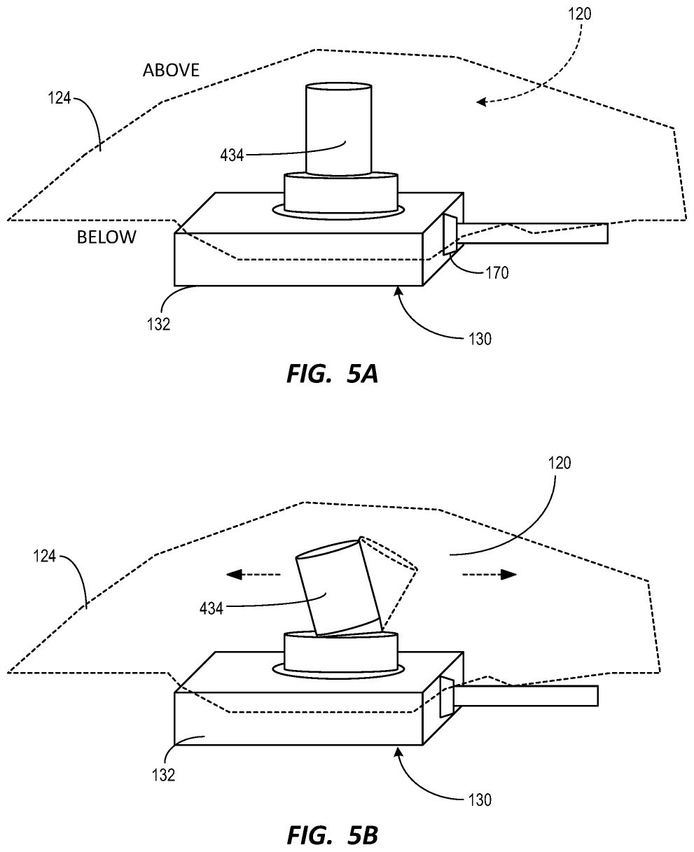

FIGS. 5A-5B illustrate an exemplary method of using the controller 130 while maintaining sterility in a sterile field 120, in accordance with some embodiments. In some embodiments, as illustrated in FIG. 5A, the controller 130 may be placed below the sterile field 120, covered with a sterile drape 124, and coupled to the computing device 110 by the computing device port 170. In some embodiments, the controller 130 includes the tactile input mechanism being the joystick 434. As illustrated in FIG. 5B, the joystick 434 may be configured to be moved in 3D space with the sterile drape 124 covering the controller 130, in order to change various parameters on the computing device 110 while maintaining sterility within the sterile field 120.

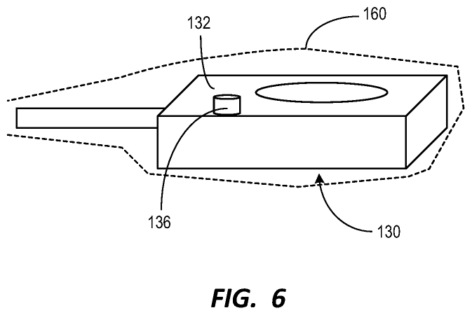

FIG. 6 illustrates a perspective view of the controller 130 within a sterile sheath 160, in accordance with some embodiments. In some embodiments, the controller 130 may be wrapped in a sterile sheath 160. In some embodiments, once the controller 130 is wrapped in the sterile sheath 160, the controller 130 may be brought into the sterile field 120 or placed below the sterile field 120. In some embodiments, the one or more controls 136 may be visually identifiable. In some embodiments, visually identifiable includes the one or more controls 136, tactile input mechanism, or non-tactile input mechanism being seen through a clear barrier. In some embodiments, the clear barrier includes the sterile sheath 160. Advantageously, the controller 130 being wrapped in a sterile sheath 160 allows the controller 130 to include the optical detection sensor and allows the user visual confirmation of physical location of the input mechanism 134 and one or more controls 136.

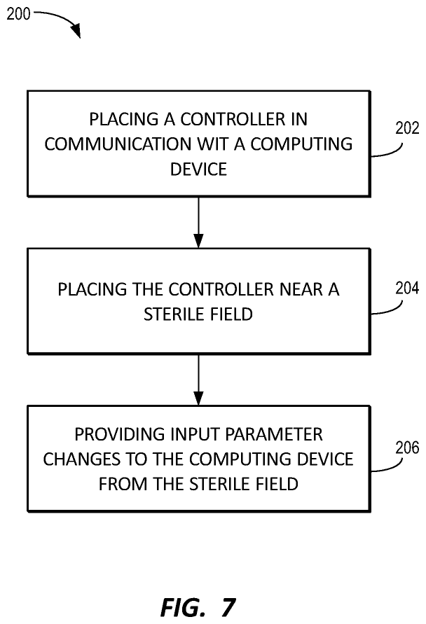

FIG. 7 illustrates a block diagram of an exemplary method of providing input parameter changes to a computing device while maintaining sterility in a sterile field, in accordance with some embodiments. In some embodiments, the method 200 includes placing the controller 130 in communication with the computing device 110 (block 202). In some embodiments, placing including connecting the controller 130 to the computing device 110. In some embodiments, placing includes placing the controller 130 in wireless communication with the computing device 110. The method 200 further includes placing the controller 130 near the sterile field 120 (block 204). In some embodiments, placing the controller 130 near the sterile field 120 includes placing the controller 130 below the sterile field 120. In some embodiments, placing the controller 130 near the sterile field 120 includes placing the controller 130 within a sterile sheath 160. In some embodiments, placing the controller 130 near the sterile field 120 includes placing the controller 130 below a sterile drape 124. The method 200 further includes providing input parameter changes to the computing device 110 (block 206). In some embodiments, providing includes providing input parameter changes through the tactile input mechanism 134 and the one or more controls 136 or through the non-tactile input mechanism 134 and the one or more controls 136. In some embodiments, the tactile input mechanism 134 may include the joystick or the directional pad and the non-tactile input mechanism 134 may include the one or more capacitive detection sensors or optical detection sensors. In some embodiments, the one or more controls 136 may include palpable controls (e.g., a knob, a button or the like). In some embodiments, the tactile input mechanism 134 and the one or more controls 136 may be visually identifiable by being seen through a clear barrier that is a sterile sheath 160 or by being illuminated.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A computing device controller system, comprising:
a computing device outside of a sterile field; and
a controller in communication with the computing device, the controller having a controller body including an input mechanism, the input mechanism including one or both of a non-tactile input and a tactile input, wherein the input mechanism is configured to be accessible in the sterile field and to provide one or more input parameter changes to the computing device.

2. The computing device controller system according to claim 1, wherein the input mechanism includes the non-tactile input, the non-tactile input comprising one or more capacitive induction sensors, one or more optical sensors, or both one or more capacitive induction sensors and one or more optical sensors.

3. The computing device controller system according to claim 1, wherein the input mechanism includes the tactile input, the tactile input comprising a joystick or a directional pad.

4. The computing device controller system according to claim 1, wherein the controller includes one or more controls configured to provide one or more input parameter changes to the computing device.

5. The computing device controller system according to claim 4, wherein the one or more controls are palpable controls.

6. The computing device controller system according to claim 5, wherein the one or more palpable controls comprise one or more of a knob, a trigger, and a button.

7. The computing device controller system according to claim 4, wherein the one or more controls are visually identifiable.

8. The computing device controller system according to claim 1, wherein the controller body includes an attachment connection port, having one or more attachment connectors configured to couple to one or more attachments within the sterile field.

9. The computing device controller system according to claim 8, wherein the one or more attachments are selected from the group consisting of an ECG module, a stylet, a magnet tracking sensor, an electromagnetic tracking sensor, an impedance driver, an impedance receiver, a fiber optic interrogator, an RFID reader, and combinations thereof.

10. The computing device controller system according to claim 8, wherein the controller is configured to transmit data from the attachment to the computing device.

11. The computing device controller system according to claim 1, wherein the sterile field is defined by a sterile drape.

12. The computing device controller system according to claim 1, wherein the controller is below the sterile field.

13. The computing device controller system according to claim 1, wherein the controller is shrouded within a sterile sheath.

14. The computing device controller system according to claim 1, wherein the controller is in wireless communication with the computing device.

15. The computing device controller system according to claim 1, wherein the one or more controls are visually identifiable through a clear barrier or by the one or more controls being illuminated.

16. The computing device controller system according to claim 1, wherein the controller is fiber optic enabled.

17. The computing device controller system according to claim 1, wherein the controller includes a console having one or more processors, non-transitory computer readable medium and a plurality of logic modules.

18. The computing device controller system according to claim 17, wherein the plurality of logic modules when activated by the one or more processors may be configured to perform one or more of:
receiving input from the non-tactile input mechanism;
correlating input from the non-tactile input mechanism with input parameter changes on the computing device;
receiving input from the one or more controls;
correlating input from the controls with input parameter changes on the computing device;
transmitting the input parameter changes to the computing device; and
illuminating the non-tactile input mechanism and controls.

19. The computing device controller system according to claim 1, wherein the computing device includes an ultrasound system.

20. The computing device controller system according to claim 1, wherein the controller includes an attachment connection port having one or more attachment connectors configured to receive one or more attachment inputs from the attachment within the sterile field.

21-32. (canceled)
